(19) United States
(12) Patent Application Publication
(10) Pub. No.: US 2002/0142999 A1
(43) Pub. Date: Oct. 3, 2002

(54) Antibiotic compositions for treatment of the eye, ear and nose

(76) Inventors: Gerald Cagle, Fort Worth, TX (US); Robert L. Abshire, Fort Worth, TX (US); David W. Stroman, Irving, TX (US); John M. Yanni, Burleson, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/092,416

(22) Filed: Mar. 6, 2002

Related U.S. Application Data
(63) Continuation of application No. 09/946,798, filed on Sep. 5, 2001.
(60) Provisional application No. 60/102,505, filed on Sep. 30, 1998.
(60) Provisional application No. 60/102,507, filed on Sep. 30, 1998.

(51) Int. Cl. A61K 31/56; A61K 31/421

(52) U.S. Cl. 514/171; 514/376

(57) ABSTRACT

Ophthalmic, otic and nasal pharmaceutical compositions containing one or more oxazolidinone antimicrobial agents are disclosed. The compositions preferably also contain one or more anti-inflammatory agents. The compositions may be utilized to treat ophthalmic, otic or nasal conditions by applying those compositions to the affected tissues.

### BACKGROUND OF THE INVENTION

The present invention is directed to the provision of topical antimicrobial compositions for the treatment of ophthalmic, otic and nasal infections, particularly bacterial infections, and to methods of treating ophthalmic, otic and nasal infections by applying those compositions to the affected tissues. The compositions and methods of the invention are based on the use of a new class of antimicrobial agents known as oxazolidinones. The compositions of the present invention may also contain one or more anti-inflammatory agents.

The use of oxazolidinones as experimental agents for the treatment of infections is described in the following publications: European Patent No. 127902, European Published Application No. 693491, European Published Application No. 127902, PCT Publication No. 9525106 and PCT Publication No. 9730995. Linezolid is an oxazolidinone under development by Pharmacia Upjohn as an antimicrobial agent which inhibits mRNA translation. Eperezolid (qv) is a similar compound also being developed by Pharmacia Upjohn.

The present invention is directed to use of oxazolidinones to treat ophthalmic, otic and nasal infections. This use of oxazolidinones is not disclosed in the above cited publications.

There is a great need for improved compositions and methods of treatment based on the use of antibacterials that are more effective than existing agents against key ophthalmic pathogens, and less prone to the development of resistance by those pathogens.

There is an even greater need for effective topical compositions and methods for treating otic and nasal infections, particularly bacterial infections. The use of oral antibacterial to treat otic infections in children has limited efficacy, and creates a serious risk of pathogen resistance to the orally administered antibacterial.

Ophthalmic, otic and nasal infections are frequently accompanied by inflammation of the infected ophthalmic, otic and nasal tissues and perhaps even surrounding tissues. Similarly, ophthalmic, otic and nasal surgical procedures that create a risk of microbial infections frequently also cause inflammation of the affected tissues. Thus, there is also a need for ophthalmic, otic and nasal pharmaceutical compositions that combine the anti-infective activity of one or more antibiotics with the anti-inflammatory activity of one or more steroid or non-steroid agents in a single composition.

### SUMMARY OF THE INVENTION

The invention is based on the use of oxazolidinone antimicrobial agents to treat ophthalmic, otic and nasal infections, as well as the prophylactic use of these antibacterial agents following surgery or other trauma to ophthalmic, otic or nasal tissues. The compositions of the present invention may also be administered to affected tissues during ophthalmic, otic or nasal surgical procedures to prevent or alleviate post-surgical infections.

The compositions preferably also contain one or more anti-inflammatory agents to treat inflammation associated with infections of ophthalmic, otic or nasal tissues. The anti-inflammatory component of the compositions is also useful in treating inflammation associated with physical trauma to ophthalmic, otic or nasal tissues, including inflammation resulting from surgical procedures. The compositions of the present invention are therefore particularly useful in treating inflammation associated with trauma to ophthalmic, otic or nasal tissues wherein there is either an infection or a risk of an infection resulting from the trauma.

Examples of ophthalmic conditions that may be treated with the compositions of the present invention include conjunctivitis, keratitis, blepharitis, dacyrocystitis, hordeolum and corneal ulcers. The compositions of the invention may also be used prophylactically in connection with various ophthalmic surgical procedures that create a risk of infection.

Examples of otic conditions that may be treated with the compositions of the present invention include otitis extema and otitis media. With respect to the treatment of otitis media, the compositions of the present invention are primarily useful in cases where the tympanic membrane has ruptured or tympanostomy tubes have been implanted. The compositions may also be used to treat infections associated with otic surgical procedures, such as tympanostomy, or to prevent such infections

The pharmacuetical compositions of the present invention are specially formulated for topical application to ophthalmic, otic and nasal tissues. The compositions are preferably sterile, and have physical properties (e.g., osmolality and pH) that are specially suited for application to ophthalmic, otic and nasal tissues, including tissues that have been compromised as the result of preexisting disease, trauma, surgery or other physical conditions.

### DETAILED DESCRIPTION OF THE INVENTION

The antimicrobial agents referred to herein as “oxazolidinones” include compounds of the following structural formula:

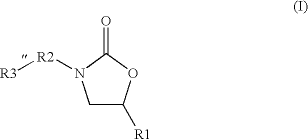

wherein:

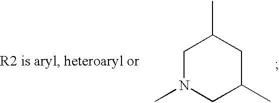

R3 is aryl, heteroaryl, C(═O)R, heterocycle or S(═O)ₙR5 wherein n=1 or 2 and R5 is alkyl or N; and

R1 is alkyl, optionally substituted by N or O, N, or a phenyl group fused onto the ring.

The following oxazolidinones are preferred in the compositions and methods of the present invention:

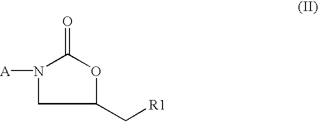

wherein:

R1 represents azido; hydroxy; or a group of the formula —OR2, —O—SO₂—R3 or —NR4R5,

wherein

R2 denotes straight-chain or branched acyl having up to 8 carbon atoms or a hydroxyl-protective group,

R3 denotes straight-chain or branched alkyl having up to 4 carbon atoms or optionally substituted wherein the substituent is a straight-chain or branched alkyl having up to 4 carbon atoms,

R4 and R5 are identical or different and denote hydrogen, or an amino-protective group, or

R4 and R5 denotes a group of the formula —CO—R6,

wherein

R6 denotes cycloalkyl having 3 to 6 carbon atoms, straight-chain or branched alkyl having up to 8 carbon atoms, phenyl or hydrogen; and

A represents a 5-membered aromatic heterocyclic radical, which has up to 3-heteroatoms selected from the group consisting of S, N or O, is directly bonded by a carbon atom and can additionally have a fused-on benzene or naphthyl ring, wherein the heterocyclic cyclic radicals are substituted in each case up to 3 times in an identical or different manner by carboxyl; halogen; cyano; mercapto; formyl; trifluoromethyl; nitro; straight-chain or branched C₁-C₆-alkoxy, straight-chain or C₁-C₆-alkoxycarbonyl; straight-chain or branched C₁-C₆-alkylthio; straight-chain or branched C₁-C₆-acyl; or optionally substituted straight-chain or branched alkyl having up to 6 carbon atoms, wherein the substituents are hydroxyl, straight-chain or branched C₁-C₅-alkoxy, C₁-C₅-acyl, or a group of the formula —NR7R8,

wherein

R7 and R8 are identical or different and denote hydrogen, straight-chain or branched alkyl having up to 4 carbon atoms or phenyl, or R7 and R8 together with the nitrogen atom form an optionally substituted 5- to 6-membered saturated heterocyclic radical which optionally has a further hetero atom selected from the group consisting of N, S or O wherein the substituents are straight-chain or branched C₁-C₂-alkyl or straight-chain or branched C₁-C₃-acyl, and/or

the heterocyclic radicals as defined in A are substituted by a group of the formula —NR7′R8′,

wherein

R7′ and R8′ are identical or different and have the abovementioned meaning of R7 and R8 and are identical to or different from these, and/or

the heterocyclic cyclic radicals as defined in A are substituted by optionally mono or disubstituted (C₁-C₈)-alkenylphenyl, optionally mono or disubstituted phenyl or by a 5- or 6-membered saturated or unsaturated mono or disubstituted heterocyclic radical having up to 3 hetero atoms selected from the group consisting of S, N or O, wherein the optional substituents are carboxyl; halogen; cyano; mercapto; formyl; trifluoromethyl; nitro; phenyl; straight-chain or branched C₁-C₆-alkoxy; straight-chain or branched C₁-C₆-alkoxycarbonyl; straight-chain or branched C₁-C₆-alkylthio, straight-chain or C₁-C₆-acyl; straight-chain or branched C₁-C₆-alkyl wherein said alkyl is optionally substituted by hydroxyl, straight-chain or branched C₁-C₅-alkoxy, straight-chain or branched C₁-C₄-acyl or a group of the formula —NR18R19,

wherein

R18 and R19 have the abovementioned meaning of R7 and R8 and are identical to or different from these; or substituted once by a group of the formula —CO—NR9R10, —NR11R12, —NR13—S(O)₂-R14, R15R16 N—SO₂— or R17—S(O)ₐ—

wherein

a denotes a number 0, 1 or 2,

R9, RIO, R13, R15 and R16 are identical or different and denote hydrogen, straight-chain or branched alkyl having up to 6 carbon atoms or phenyl,

R11 and R12 are identical or different and have the abovementioned meaning of R7 and R8 and are identical or different from these,

R14 and R17 are identical or different and have the abovementioned meaning of R3 and are identical to or different from this, and/or

the heterocyclic cyclic radicals are substituted by a radical of the formula

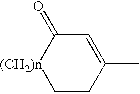

wherein n denotes the number 0, 1 or 2;

or a salt or S-oxide thereof.

The oxazolidinones of formula (I) and formula (II) above are known compounds. Further details regarding the structure, preparation, and physical properties of oxazolidinones of formula (II) are provided in U.S. Pat. No. 5,698,574.

The concentrations of the oxazolidinones in the compositions of the present invention will vary depending on the intended use of the compositions (e.g., treatment of existing infections or prevention of post-surgical infections), and the relative antimicrobial activity of the specific oxazolidinone. The activity of antimicrobials is generally expressed as the minimum concentration of a compound required to inhibit the growth of a specified pathogen. This concentration is also referred to as the “minimum inhibitory concentration” or “MIC”. The term “MIC90” refers to the minimum concentration of an antimicrobial compound required to inhibit the growth of ninety percent (90%) of the strains of a species. The concentration of a compound required to totally kill a specified bacteria is referred to as the “minimum bactericidal concentration” or “MC”.

The appropriate concentration for ophthalmic compositions will generally be an amount of oxazolidinone sufficient to provide a concentration in the aqueous humor and lacrimal fluid of the eye equal to or greater than the MIC 90 level for the selected oxazolidinone, relative to gram-negative and gram-positive organisms commonly associated with ophthalmic infections. The appropriate concentrations for otic and nasal compositions will generally be an amount of one or more antibiotics of formula (I) sufficient to provide a concentration in the infected tissues equal to or greater than the MIC90 level for the selected antibiotic(s), relative to gram-negative and gram-positive organisms commonly associated with otic or nasal infections. Such an amount is referred to herein as “an antimicrobial effective amount”. The compositions of the present invention will typically contain one or more oxazolidinones in a concentration of from about 0.1 to about 1.0 percent by weight (“wt %”) of the compositions.

The compositions of the present invention may also contain one or more anti-inflammatory agents. The anti-inflammatory agents utilized in the present invention are broadly classified as steroidal or non-steroidal. The preferred steroidal anti-inflammatory agents are glucocorticoids.

The preferred glucocorticoids for ophthalmic and otic use include dexamethasone, loteprednol, rimexolone, prednisolone, fluorometholone, and hydrocortisone. The preferred glucocorticoids for nasal use include mometasone, fluticasone, beclomethasone, flunisolide, triamcinolone and budesonide.

The dexamethasone derivatives described in U.S. Pat. No. 5,223,493 (Boltralik) are also preferred steroidal anti-inflammatory agents, particularly with respect to compositions for treating ophthalmic inflammation. The following compounds are especially preferred:

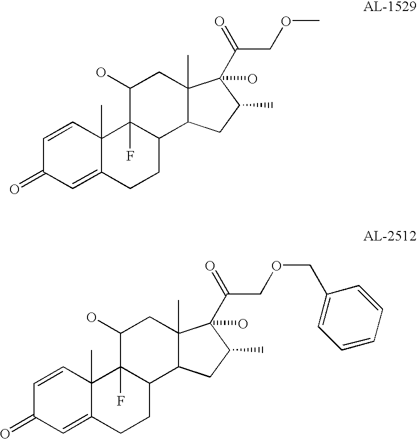

These compounds are referred to herein as “21-ether derivatives of dexamethasone”. The 21-benzyl ether derivative (i.e., compound AL-2512) is particularly preferred.

The preferred non-steroidal anti-inflammatory agents are: prostaglandin H synthetase inhibitors (Cox I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NFkB transcription factor; or other anti-inflammatory agents known to those skilled in the art.

The concentrations of the anti-inflammatory agents contained in the compositions of the present invention will vary based on the agent or agents selected and the type of inflammation being treated. The concentrations will be sufficient to reduce inflammation in the targeted ophthalmic, otic or nasal tissues following topical application of the compositions to those tissues. Such an amount is referred to herein as “an anti-inflammatory effective amount”. The compositions of the present invention will typically containe one or more anti-inflammatory agents in an amount of from about 0.01 to about 1.0 wt. %.

The compositions of the present invention are typically administered to the affected ophthalmic, otic or nasal tissues by topically applying one to four drops of a sterile solution or suspension, or a comparable amount of an ointment, gel or other solid or semisolid composition, one to four times per day. However, the compositions may also be formulated as irrigating solutions that are applied to the affected ophthalmic, otic or nasal tissues during surgical procedures.

The ophthalmic, otic and nasal compositions of the present invention will contain one or more oxazolidinones in pharmaceutically acceptable vehicles. The compositions will typically have a pH in the range of 4.5 to 8.0. The ophthalmic compositions must also be formulated to have osmotic values that are compatible with the aqueous humor of the eye and ophthalmic tissues. Such osmotic values will generally be in the range of from about 200 to about 400 milliosmoles per kilogram of water (“mOsm/kg”), but will preferably be about 300 mOsm/kg.

Ophthalmic, otic and nasal products are typically packaged in multidose form. Preservatives are thus required to prevent microbial contamination during use. Suitable preservatives include: polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g., Pluronic F-68, F-84 and P-103), cyclodextrin, or other agents known to those skilled in the art. Typically such co-solvents are employed at a level of from 0.01% to 2% by weight.

The use of viscosity enhancing agents to provide the compositions of the invention with viscosities greater than the viscosity of simple aqueous solutions may be desirable to increase absorption of the active compounds by the target tissues or increase the retention time in the eye, ear or nose. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

The following examples are provided to further illustrate the ophthalmic, otic and nasal compositions of the present invention.

### EXAMPLE 1

### Ophthalmic/Otic/Nasal Solution

| Ingredient | Amount (wt. %) |
|---|---|
| Oxazolidinone | 0.35 |
| Sodium Acetate | 0.03 |
| Acetic Acid | 0.04 |
| Mannitol | 4.60 |
| EDTA | 0.05 |
| Benzalkonium Chloride | 0.006 |
| Water | q.s.100 |

### EXAMPLE 2

### Ophthalmic/Otic/Nasal Suspension

| Ingredient | Amount (wt. %) |
|---|---|
| Oxazolidinone | 0.3 |
| Dexamethasone, Micronized USP | 0.10 |
| Benzalkonium Chloride | 0.01 |
| Edetate Disodium, USP | 0.01 |
| Sodium Chloride, USP | 0.3 |
| Sodium Sulfate, USP | 1.2 |
| Tyloxapol, USP | 0.05 |
| Hydroxyethylcellulose | 0.25 |
| Sulfuric Acid and/or | q.s. for pH adjustment to 5.5 |
| Sodium Hydroxide, NF | |
| Purified Water, USP | q.s. to 100 |

### EXAMPLE 3

### Ophthalmic Ointment

| Ingredient | Amount (wt. %) |
|---|---|
| Oxazolidinone | 0.35 |
| Mineral Oil, USP | 2.0 |
| White petrolatium, USP | q.s 100 |

### EXAMPLE 4

### Ophthalmic Ointment

| Ingredient | Amount (wt. %) |
|---|---|
| Oxazolidinone | 0.3 |
| Fluorometholone Acetate, USP | 0.1 |
| Chlorobutanol, Anhydrous, NF | 0.5 |
| Mineral Oil, USP | 5 |
| White Petrolatum, USP | q.s. 100 |

The invention has been described herein by reference to certain preferred embodiments. However, as obvious variations thereon will become apparent to those skilled in the art, the invention is not to be considered as limited thereto.

What is claimed is:

1. A topical ophthalmic, otic or nasal pharmaceutical composition comprising an antimicrobial effective amount of an oxazolidinone and a phannacuetically acceptable vehicle therefor.

2. A topical composition according to claim 1, wherein the composition further comprises an anti-inflammatory effective amount of a steroidal or non-steroidal anti-inflammatory agent.

3. A topical composition according to claim 2, wherein the anti-inflammatory agent comprises a glucocorticoid.

4. A topical composition according to claim 3, wherein the glucocorticoid is is selected from the group consisting of dexamethasone, rimexolone, prednisolone, fluorometholone, hydrocortisone, mometasone, fluticasone, beclomethasone, flunisolide, triamcinolone and budesonide.

5. A topical composition according to claim 2, wherein the anti-inflammatory agent comprises a non-steroidal agent selected from the group consisting of prostaglandin H synthetase inhibitors, PAF antagonists, and PDE IV inhibitors.

6. A method of treating or preventing ophthalmic, otic or nasal infections, which comprises topically applying a therapeutically effective amount of the composition of claim 1 to the affected ophthalmic, otic or nasal tissue.

7. A method of treating or preventing ophthalmic, otic or nasal infections and attendant inflammation, which comprises topically applying a therapeutically effective amount of the composition of claim 2 to the affected ophthalmic, otic or nasal tissue.
